(19) United States
(12) Patent Application Publication
(10) Pub. No.: US 2017/0224428 A1
(43) Pub. Date: Aug. 10, 2017

(54) DYNAMIC INPUT SCALING FOR CONTROLS OF ROBOTIC SURGICAL SYSTEM

(71) Applicant: Covidien LP, Mansfield, MA (US)

(72) Inventor: Brock Kopp, Branford, CT (US)

(73) Assignee: Covidien LP, Mansfield, MA (US)

(21) Appl. No.: 15/514,915

(22) PCT Filed: Sep. 21, 2015
(86) PCT No.: PCT/US2015/051130

§ 371 Date: Mar. 28, 2017

Related U.S. Application Data
(60) Provisional application No. 62/056,767, filed on Sep. 29, 2014.

(51) Int. Cl. A61B 34/30; A61B 34/00; B25J 9/16

(52) U.S. Cl. CPC A61B 34/30; B25J 9/1646; A61B 34/77; A61B 34/74; G05B 2219/42352

(57) ABSTRACT

A robotic surgical system includes an arm, a tool, an input controller, and a processing unit. The arm includes an end that supports the tool which is moveable an output distance within a surgical site. The input controller is movable an input distance at an input velocity and acceleration. The processing unit is in communication with the input controller and is operatively associated with the arm to move the tool the output distance. The processing unit is configured to dynamically scale the output distance in response to the input distance, velocity, and/or acceleration.

### CROSS-REFERENCE TO RELATED APPLICATIONS

This application is a U.S. National Stage Application filed under 35 U.S.C. §371(a) of International Patent Application Serial No. PCT/US2015/051130, filed Sep. 21, 2015, which claims the benefit of and priority to U.S. Provisional Patent Application Ser. No. 62/056,767, filed Sep. 29, 2014, the entire disclosure of which is incorporated by reference herein.

### BACKGROUND

Robotic surgical systems have been used in minimally invasive medical procedures. During a medical procedure, a surgeon moved an input controller of the robotic surgical system to control a robot arm and surgical instrument attached thereto. The input controller was moveable within a limited range of motion to control the movement of the robot arm and/or surgical tool. When the input controller reached a limit of this range of motion the surgeon decoupled or “clutched out” the movement of the input controller from the movement of the robot arm to continue moving the tool in the same direction.

One advantage of a robotic surgical system was the ability to scale down the movement of the input controller. A large movement of the input controller was reduced to a smaller movement of the surgical tool. This scaling down of movement allowed the surgeon to be more precise during a robotic surgical procedure than a traditional surgical procedure. The Output_{distance} (i.e., movement of the robotic system) was scaled down by the Input_{distance} (i.e., movement of the input controller) using a scaling factor S_{f} (i.e., Output_{distance}=Input_{distance}/S_{f}). The scaling down of movement also minimized small jitters, shaking, or tremors in the movement of the surgeon.

A disadvantage of scaling down the movement of the input controller was the exacerbation of the limited range of motion of the input controller. As the scaling factor increased, the surgeon was required to “clutch out” more frequently as the input controller had to be moved further for the tool to travel a similar distance and therefore reached a limit in its range of motion faster.

There is a need for robotic surgical system that scales down the movement of the surgeon while reducing instances of a surgeon reaching an end of a range of motion of the input controller and having to “clutch out” during robotic surgical procedures.

### SUMMARY

A robotic surgical system may include a robotic arm supporting a surgical tool, an input controller movable in at least three dimensions, a sensor, and a processing unit. The sensor may detect a movement distance, velocity, and/or acceleration of the input controller as the input controller is moved in the at least three dimensions. The processing unit may be operatively associated with the robotic arm to move the tool an output distance. The processing unit may also be configured to dynamically scale the movement distance based on the movement velocity or acceleration and calculate the output distance from the dynamic scaling.

The sensor may be configured to send signals indicative of the movement distance, velocity, and/or acceleration of the input controller to the processing unit. The processing unit may be configured to calculate the output distance in different way. For example, the processing unit may calculate the output distance by multiplying the movement distance by the movement velocity or the processing unit may calculate the output distance by multiplying the movement distance by a predetermined scaling factor that varies depending the movement velocity and/or acceleration. The predetermined scaling factor may be a first value when the movement velocity and/or acceleration are within a first range and a second value when the velocity and/or acceleration are within a second range different from the first range.

The processing unit may be configured to scale the movement distance by a distance, velocity, and/or acceleration scaling factor. The scaling factor(s) may be constant or may vary. At least one of the scaling factors may be changeable before or during a surgical procedure. At least one of the scaling factors may be in a range of about 1 to about 10 in some instances, but in other instances the range may be different. The processing unit may be configured to scale the output distance to the product of the input distance over the distance scaling factor and the input velocity over the velocity scaling factor.

The robotic surgical system may also include a motor in communication with the processing unit. The motor may be configured to move the robotic arm in response to a scaled control signal received from the processing unit.

A method of operating a surgical robot may include moving a tool of a robotic surgical system an output distance that is dynamically scaled by a processing device based on at least one of a distance, speed, and acceleration at which an input controller is moved. A control signal indicative of a distance, speed, and/or acceleration at which the input controlled is moved may be sent to the processing unit. A scaled control signal may be sent to an arm of the robotic surgical system to move the tool the output distance.

Dynamically scaling the control signal may include dividing the input velocity by a velocity scaling factor. Additionally or alternatively, dynamically scaling the control signal may include dividing the input distance by a distance scaling factor, calculating the output distance from the product of the input distance over a distance scaling factor and the input velocity over a velocity scaling factor, and/or adjusting at least one of the distance scaling factor or the velocity scaling factor.

### BRIEF DESCRIPTION OF THE DRAWINGS

Various aspects of the present disclosure are described with reference to the drawings, which are incorporated in and constitute a part of this specification, wherein:

FIG. 1 is a schematic illustration of a user interface and a robotic console.

FIG. 2 shows exemplary methods

### DETAILED DESCRIPTION

A scaling factor that scales down movement of the input controller may be dynamically adjusted as the input controller is moved by a user during surgery. The dynamic adjustment of the scaling factor may be based on a speed or acceleration at which the user moves the input controller. If the user moves the input controller more quickly, then the scaling factor may be reduced so that the associated robotic arm and/or surgical tool moves proportionately further than if the user moved the input controller at a slower speed. If the user moves the input controller slower, then the scaling factor may be increased so that the associated robotic arm and/or surgical tool move proportionately less than at the faster speed. Dynamically adjusting the scaling factor reduces the number of times a user reaches an end of a range of motion of the input controller by moving the surgical tool proportionately further distances the fast the input controller is moved.

A clinician may include a doctor, a nurse, or any other care provider and may include support personnel. A proximal portion of a device or component may refer to a portion that is closest to a clinician and/or closer to the clinician than a distal portion, which may be located farthest from the clinician.

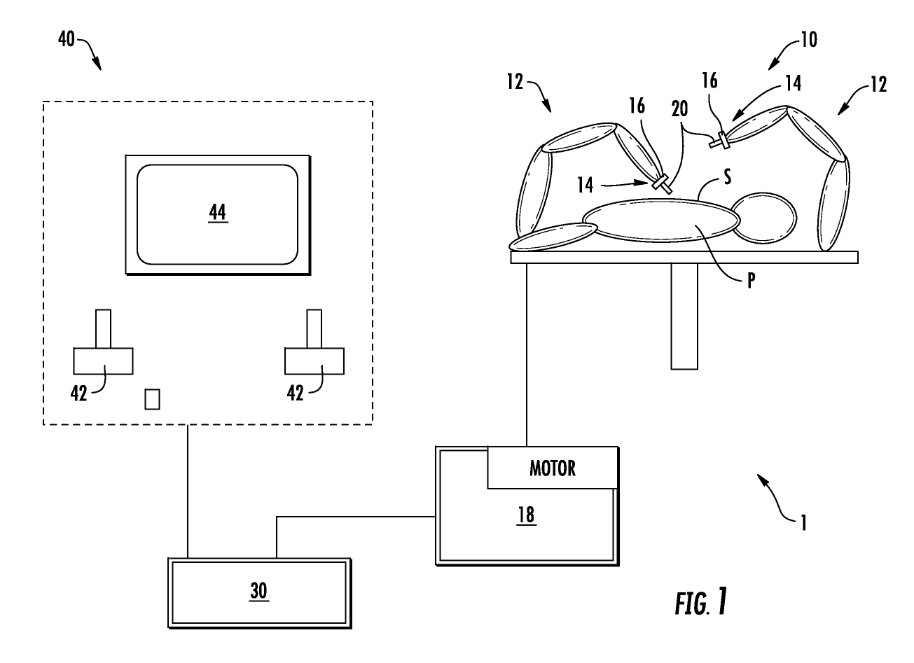

Referring to FIG. 1, a robotic surgical system 1 in accordance with the present disclosure is shown generally as a robotic system 10, one or more sensors 11, a processing unit 30, and a user interface 40. The robotic system 10 generally includes a plurality of arms 12 and a robot base 18. An end 14 of each of the arms 12 supports an end effector or tool 20 which is configured to act on tissue. In addition, the ends 14 of the arms 12 may include an imaging device 16 for imaging a surgical site “S” adjacent the tool 20. The user interface 40 is in communication with robot base 18 through the processing unit 30.

The user interface 40 includes a display device 44 which is configured to display images. In some instances, the display device 44 may display two- or three-dimensional images of the surgical site “S” which may include data captured by imaging devices 16 positioned on the ends 14 of the arms 12 and/or include data captured by imaging devices (not shown) that are positioned about the surgical theater (e.g., an imaging device positioned within the surgical site “S”, an imaging device positioned adjacent the patient “P”). The imaging devices (e.g., imaging device 16) may capture visual images, infra-red images, ultrasound images, X-ray images, thermal images, and/or any other known real-time images of the surgical site “S”. The imaging devices transmit captured imaging data to the processing unit 30 which creates the three-dimensional images of the surgical site “S” in real-time from the imaging data and transmits the three-dimensional images to the display device 44 for display. Imaging devices 16 may be tools 20 or otherwise integrated with the tools 20.

The user interface also includes input controllers 42 which allow a surgeon to manipulate the robotic system 10 (e.g., move the arms 12, the ends 14 of the arms 12, and/or the tools 20). Each of the input controllers 42 is in communication with the processing unit 30 to transmit control signals thereto and to receive feedback signals therefrom. Additionally or alternatively, each of the input controllers 42 may include control interfaces (not shown) which allow the surgeon to manipulate (e.g., clamp, grasp, fire, open, close, rotate, thrust, slice, etc.) the tools 20 supported at the ends 14 of the arms 12.

An input controller 42 may include one or more sensors 11. A sensor 11 may detect a movement distance and/or a movement velocity of the input controller as the input controller is moved in the at least three dimensions. In some instances, a sensor 11 may be integrated into the input controller 42, but in other instances, a sensor 11 may be located away from the input controller 42. For example, a position sensing detector or an image sensor such as a CCD or CMOS sensor may be directed toward a portion of the input controller 42 to detect a movement distance and/or speed of the input controller without necessarily being located on or in the input controller 42.

Each of the input controllers 42 is moveable through a predefined three-dimensional range of motion to move the tools 20 within a surgical site “S.” The three-dimensional images on the display device 44 are orientated such that the movement of the input controller 42 moves the tools 20 as viewed on the display device 44. It will be appreciated that the orientation of the three-dimensional images on the display device may be mirrored or rotated by the clinician to a desired viewing orientation to permit the surgeon to have a better view or orientation to the surgical site “S”. In addition, it will be appreciated that the size of the three-dimensional images on the display device 44 may be scaled to be larger or smaller than the actual structures of the surgical site permitting the surgeon to have a better view of structures within the surgical site “S”. As the input controllers 42 are moved, the tools 20 are moved within the surgical site “S” as detailed below. As detailed herein, movement of the tools may include moving the ends 14 of the arms 12 which support the tools 20.

For a detailed discussion of the construction and operation of a robotic surgical system 1, reference may be made to U.S. Patent Publication No. 2012/0116416, filed on Nov. 3, 2011, entitled “Medical Workstation,” the entire contents of which are incorporated herein by reference.

The movement of the tools 20 is scaled relative to the movement of the input controllers 42. When the input controllers 42 are moved within the predefined range of motion, the input controllers 42 send control signals to the processing unit 30. The processing unit 30 analyzes the control signals to move the tools 20, in response to the control signals. The processing unit 30 transmits scaled control signals to the robot base 18 to move the tools 20 in response to the movement of the input controllers 42. The processing unit 30 scales the control signals by dividing an Input_{distance} (e.g., the distance moved by one of the input controllers 42) by a distance scaling factor DS_{f} to arrive at a scaled Output_{distance} (e.g., the distance that one of the tools 20 is moved). In some instances, the distance scaling factor DS_{f} is in a range between about 1 and about 10 (e.g., 3), but in other instances, other scaling factors may be used. This portion of the scaling equation is represented by the following equation:

Output_{distance}=Input_{distance}/DS_{f}

It will be appreciated that the larger the distance scaling factor DS_{f} the smaller the movement of the tools 20 relative to the movement of the input controllers 42.

During a surgical procedure, if the surgeon reaches the edge or limit of the predefined range of motion of an input controller 42, the surgeon must clutch the input controller 42 (i.e., reposition the input controller 42 back within the predefined range of motion) before continuing to move the input controller 42 in the same direction. The surgeon may be required to clutch the input controller 42 one or more times to complete a single action (e.g., cutting a structure within the surgical site “S”) during a surgical procedure. As the distance scaling factor DS_{f} is increased, the surgeon may be required to clutch the input controller 42 more frequently, which increases the number of steps and thus, the time and/or costs of the surgical procedure.

To reduce the number of times a surgeon is required to clutch the input controllers 42 to perform a single action and the number of times a surgeon is required to clutch during a surgical procedure, the processing unit 30 may dynamically scale the control signals to account for an Input_{velocity} (e.g., the speed and/or acceleration at which the input controllers 42 are moved). In some instances, the control signals may be dynamically scaled to account for an acceleration of the input controllers in addition to or instead of the velocity. Thus, the term input Input_{velocity} may refer to a speed at which the input controllers 42 are moved, an acceleration at which the input controllers 42 are moved, or both a speed and acceleration at which the input controllers 42 are moved. The processing unit 30 may dynamically scale the Input_{velocity} by a velocity scaling factor VS_{f} and multiply the result by the result of the Input_{distance} divided by the distance scaling factor DS_{f}. In some instances, the velocity scaling factor VS_{f} is in a range between about 1 and about 10 (e.g., 1.5, 2, or 3), but in other instances other scaling factors may be used. This dynamic scaling may be represented by the following equation:

Output_{distance}=(Input_{distance}/DS_{f})*(Input_{velocity}/VS_{f})

It will be appreciated that the larger the velocity scaling factor VS_{f} the less the velocity will affect the Output_{distance}.

Including the Input_{velocity} in the scaling of the movement of the ends 14 of the arms 12 allows for dynamic scaling of the movement of the ends 14. The dynamic scaling allows the surgeon to perform small precise motions while also being able to move a large distance quickly without clutching. In addition, actions that benefit from a single continuous stroke (e.g., cutting) may be completed with a single uninterrupted action over a large distance. For example, where an uninterrupted action is taking place over a relatively constant velocity.

It will be appreciated that while the Input_{distance} is dynamically scaled to the Output_{distance} based on the distance and velocity that the input controllers 42 are moved, the scaling factor DS_{f} and the velocity scaling factor VS_{f} may remain constant during a single action. In some instances, the distance scaling factor DS_{f} and the velocity scaling factor VS_{f} may be initially fixed at the time of manufacturing or programming of the processing unit 30, and then may be selectively switched into a dynamically adjustable mode prior to each surgical procedure, or may be selectively switched into the dynamically adjustable mode by the surgeon during the surgical procedure.

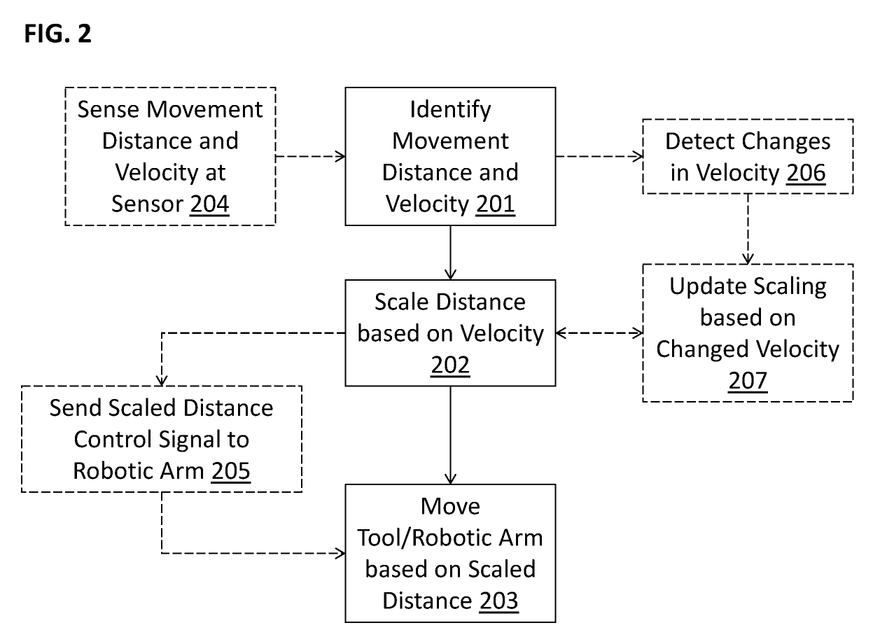

FIG. 2 shows an exemplary method of operating a surgical robot. In box 201, a movement distance, velocity, and/or acceleration of an input controller of a robotic surgical system moveable in at least three dimensions is identified. In box 204, the movement distance, velocity, and/or acceleration of the input controller may be sensed from one or more sensors that may be integrated into the input controller or separate from the input controlled.

In box 202, the identified movement distance is dynamically scaled based on at least one of the identified movement velocity and acceleration. In box 205, a control signal based on the dynamically scaled movement distance may be sent to the robotic arm. The dynamic scaling may include one or more of the algorithms discussed herein and/or other algorithms. For example, dynamic scaling may include multiplying the identified movement distance by the identified movement velocity and/or acceleration. The dynamic scaling may also include dividing the identified movement velocity by a velocity scaling factor. The dynamic scaling may also include dividing the identified movement distance by a distance scaling factor. At least one of the distance scaling factor or the velocity scaling factor may be adjusted based on a predetermined criterion. The criterion may include a type of tool attached to a robotic arm, a type of robotic arm coupled to the input controller, a user selected function or feature associated with a predetermined scaling factor, or other predetermined criterion.

The dynamic scaling may include calculating a product of the movement distance divided by a distance scaling factor and the movement velocity and/or acceleration divided by a velocity scaling factor.

In box 203, a surgical tool coupled to a robotic arm is moved based on the dynamically scaled movement distance. In some instances, the robotic arm may be moved based on the control signal received at the robotic arm, the moving of the robotic arm moving the surgical tool.

In box 206, two or more different movement velocities of the input controller may be detected over a predetermined time. This may occur if a user changes the speed at which they are moving the input controller by, for example, suddenly accelerating or decelerating. In box 207, the scaling of the movement distance may be dynamically updated for each of the respective detected movement velocity changes. In some instances, the surgical tool may be moved by different relative amounts according to the updated dynamic scaling, so that the relative movement amount changes as a dynamic scaling value changes.

While several embodiments of the disclosure have been shown in the drawings, it is not intended that the disclosure be limited thereto, as it is intended that the disclosure be as broad in scope as the art will allow and that the specification be read likewise. Any combination of the above embodiments is also envisioned and is within the scope of the appended claims. Therefore, the above description should not be construed as limiting, but merely as exemplifications of particular embodiments. Those skilled in the art will envision other modifications within the scope of the claims appended hereto.

What is claimed:

1. A robotic surgical system comprising:
a robotic arm supporting a surgical tool;
an input controller movable in at least three dimensions;
a sensor detecting a movement distance and at least one of a movement velocity and an acceleration of the input controller as the input controller is moved in the at least three dimensions; and
a processing unit operatively associated with the robotic arm to move the tool an output distance, configured to dynamically scale the movement distance based on at least one of the movement velocity and the acceleration, and configured to calculate the output distance from the dynamic scaling.

2. The system of claim 1, wherein the sensor is configured to send signals indicative of the movement distance and the movement velocity of the input controller to the processing unit.

3. The system of claim 1, wherein the processing unit is configured to calculate the output distance by multiplying the movement distance by the movement velocity.

4. The system of claim 1, wherein the processing unit is configured to calculate the output distance by multiplying the movement distance by a predetermined scaling factor that varies depending on at least one of the movement velocity and the acceleration.

5. The system of claim 4, wherein the predetermined scaling factor is a first value when the movement velocity or the acceleration is within a first range and a second value when within a second range different from the first range.

6. The system of claim 1, wherein the processing unit is configured to scale the movement distance by a distance scaling factor and a velocity scaling factor.

7. The system of claim 6, wherein the distance scaling factor and the velocity scaling factor are constant.

8. The system of claim 6, wherein at least one of the distance scaling factor and the velocity scaling factor is changeable before or during a surgical procedure.

9. The system of claim 8, wherein at least one of the distance scaling factor and the velocity scaling factor is in a range of about 1 to about 10.

10. The system of claim 6, wherein the processing unit is configured to scale the output distance to the product of the input distance over the distance scaling factor and the input velocity over the velocity scaling factor.

11. The system of claim 1, further comprising a motor in communication with the processing unit, the motor configured to move the robotic arm in response to a scaled control signal received from the processing unit.

12. A method of operating a surgical robot, the method comprising:
identifying a movement distance and movement velocity of an input controller of a robotic surgical system moveable in at least three dimensions;
dynamically scaling the identified movement distance based on the identified movement velocity; and
moving a surgical tool coupled to a robotic arm based on the dynamically scaled movement distance.

13. The method of claim 12, further comprising sensing the movement distance and movement velocity of the input controller from one or more sensors.

14. The method of claim 12, further comprising:
sending a control signal based on the dynamically scaled movement distance to the robotic arm; and
moving the robotic arm based on the control signal received at the robotic arm, the moving of the robotic arm moving the surgical tool.

15. The method of claim 12, further comprising multiplying the identified movement distance by the identified movement velocity as part of the dynamic scaling.

16. The method of claim 15, further comprising dividing the identified movement velocity by a velocity scaling factor as part of the dynamic scaling.

17. The method of claim 16, further comprising dividing the identified movement distance by a distance scaling factor as part of the dynamic scaling.

18. The method of claim 17, further comprising adjusting at least one of the distance scaling factor or the velocity scaling factor based on a predetermined criterion.

19. The method of claim 12, further comprising calculating a product of the movement distance divided by a distance scaling factor and the movement velocity divided by a velocity scaling factor as part of the dynamic scaling.

20. The method of claim 12, further comprising:
detecting a plurality of changes in the identified movement velocity of the input controller;
updating the dynamic scaling for at least two of the detected movement velocity changes; and
moving the surgical tool by different relative amounts according to the update dynamic scaling.
